(19) United States
(12) Patent Application Publication
(10) Pub. No.: US 2026/0160679 A1
(43) Pub. Date: Jun. 11, 2026

(54) AUTOMATED METHOD FOR ESTIMATING THE DEGRADATION OF AN ACTIVE SUBSTANCE

(71) Applicant: UNIVERSIDAD SIMÓN BOLÍVAR, Barranquilla, Atlántico (CO)

(72) Inventors: Reynaldo VILLARREAL GONZÁLEZ, Barranquilla, Atlántico (CO); Nataly GALÁN FREYLE, Barranquilla, Atlántico (CO); Leonardo PACHECO LONDOÑO, Barranquilla, Atlántico (CO); Samuel HERNÁNDEZ RIVERA, Mayaguez, PR (US)

(21) Appl. No.: 19/104,086

(22) PCT Filed: Aug. 16, 2023
(86) PCT No.: PCT/CO2023/000015

§ 371 Date: Feb. 14, 2025

(30) Foreign Application Priority Data
Aug. 17, 2022 (CO) NC2022/0011592

(51) Int. Cl. G01N 21/3563; G01N 33/15

(52) U.S. Cl. CPC G01N 21/3563; G01N 33/15; G01N 2201/06; G01N 2201/129

(57) ABSTRACT

The present solution consists of a method that allows the determination of the quantity and content uniformity of an active ingredient in a pharmaceutical formulation. This method involves reading the infrared spectroscopic signal of the active ingredient, preferably using a quantum cascade laser (QCL), although other spectroscopic techniques are also valid.
The spectroscopic data obtained is analyzed through a multivariable analysis model using partial least squares (PLS), which is then used to train an artificial intelligence system. This trained AI is capable of determining the concentration of the active ingredient in the pharmaceutical formulation.
By taking measurements at different physical points in the formulation, it is also possible to determine the distribution of the active ingredient within the formulation, and therefore, its uniformity.

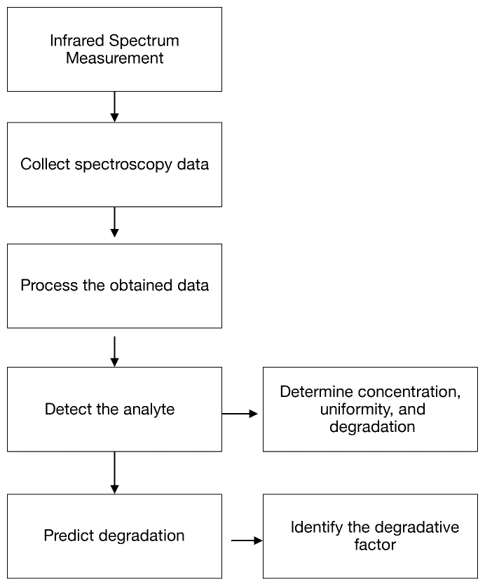

### FIELD OF THE INVENTION

The following invention belongs to the field of pharmaceutical chemistry, specifically related to the uniformity of active ingredients in formulations.

### STATE OF THE ART

Within the state of the art, certain prior technical solutions relevant to this invention were identified. Among them, the most relevant is described below.

US2008228428 ‘System and Method for the Non-Destructive Determination of the Quantitative Spatial Distribution of Components in a Medical Product’ describes a method and system for non-destructive analysis of medical devices. It uses a confocal Raman microscope and other non-destructive analytical tools to assess the spatial distribution of components within an object, such as the distribution of an active pharmaceutical ingredient (API) within a polymer matrix.

This method includes the use of Raman spectroscopy and near-infrared instruments. However, these techniques are limited by signal overlap from excipients and impurities in the formulation, making it difficult to determine the level of degradation of the active ingredient.

### BRIEF DESCRIPTION OF THE INVENTION

The present solution consists of a method that enables the determination of the quantity and content uniformity of an active ingredient in a pharmaceutical formulation. The method involves reading the infrared spectroscopic signal of the active ingredient, preferably using a quantum cascade laser (QCL), although other spectroscopic techniques are also valid.

### DETAILED DESCRIPTION OF THE INVENTION

The problem addressed by this invention is the difficulty of determining the concentration and uniformity of distribution of an active ingredient within a pharmaceutical formulation without destroying it.

This solution employs a method that combines the use of a quantum cascade laser (QCL) with data analysis through a partial least squares (PLS) model, implemented within a data processing module using artificial intelligence. This approach enables analysis without sample preparation, reduces analysis time, and enhances sensitivity.

To perform the process, infrared spectroscopy measurements are taken at multiple locations on the surface of the formulation, with a minimum of eight measurements to maximize coverage. In a preferred embodiment, twenty measurements are performed.

The collected spectroscopic data is analyzed using the data processing module, which applies a PLS model to predict the concentration and distribution of the active ingredient by comparing concentration levels across different areas of the formulation.

The artificial intelligence system has been pre-trained with data from formulations with varying concentrations of the active ingredient. It uses machine learning, specifically random forest models, to classify degradation products and determine the cause of degradation (e.g., temperature, humidity, etc.).

The data processing module outputs percentage values reflecting the concentration and distribution of the active ingredient in the formulation.

What is claimed is:

1. An automated method for determining concentration, content uniformity, and degradation of the active ingredient in pharmaceutical formulations, comprising:
a) Measuring the infrared spectrum at at least eight points on the surface of the formulation;
b) Collecting spectroscopic data;
c) Analyzing the obtained data using a data processor that integrates artificial intelligence;
d) Detecting the analyte and determining its concentration, content uniformity, and degradation in the analyzed formulation;
e) Predicting the presence of degradation and, if applicable, proceeding to the next phase;
f) Identifying the degradation factor.

2. The method of claim 1, where the spectroscopic measurement must be performed using a quantum cascade laser (QCL) or any other high-power spectroscopic method.

3. The method of claim 1, where the artificial intelligence concentration analysis is performed through a partial least squares (PLS) regression, allowing for the determination of the active ingredient concentration level.

4. The method of claim 1, where the prediction of uniformity in the formulation is based on a comparison between the spectra of the analyzed surface zones.

5. The method of claim 1, where the determination of the degradation level is performed by subtracting the base spectrum from the analyzed spectrum using the formula:

6. The method of claim 1, where the identification of the degradation factor is performed using a random forest machine learning model that classifies previously trained factors.

7. The process of claim 1, where all data processing is carried out immediately by a cloud server.
